(19) United States
(12) Patent Application Publication
(10) Pub. No.: US 2024/0416119 A1
(43) Pub. Date: Dec. 19, 2024

(54) POWER LINK OPTIMIZATION VIA AN INDEPENDENT DATA LINK

(71) Applicant: Cochlear Limited, Macquarie University, NSW (AU)

(72) Inventors: Helmut Christian EDER, Bemboka, NSW (AU); Hans VANDENWIJNGAERDEN, West Ryde, NSW (AU)

(21) Appl. No.: 18/703,172

(22) PCT Filed: Oct. 18, 2022
(86) PCT No.: PCT/IB2022/059990

§ 371 Date: Apr. 19, 2024

Related U.S. Application Data
(60) Provisional application No. 63/272,220, filed on Oct. 27, 2021.

(51) Int. Cl. A61N 1/36; A61N 1/378

(52) U.S. Cl. CPC A61N 1/36038; A61N 1/3787

(57) ABSTRACT

Presented herein are devices, systems, and methods for transmitting power to an implantable component via a power link, and receiving data from the implantable component via a data link that is separate from the power link. The data received from the implantable component indicates a power requirement of the implantable component, which may be used to regulate the power transmitted via the power link.

### BACKGROUND

### Field of the Invention

The present invention relates generally to techniques for optimizing a transcutaneous power link in an implantable medical device system.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: transmitting power from an external device to an implantable medical device via a power link; and receiving, at the external device, data from the implantable medical device via a data link that is separate from the power link, wherein the data indicates a real-time power requirement of the implantable medical device.

In another aspect, a method is provided. The method comprises: transmitting power signals from an external component to an implantable component via a first transcutaneous link; receiving, at the external component, power-regulation data from the implantable component via a second transcutaneous link that is different from the first transcutaneous link; and using the power-regulation data to regulate transmission of the power signals from the external component to the implantable component.

In another aspect, an apparatus is provided. The apparatus comprises: a radio-frequency module configured to send power to an implantable medical device via a closely-coupled power link; a wireless module configured to receive power-regulation data from the implantable medical device, where the power-regulation data is received via a data link that is separate from the closely-coupled power link; and an external power regulator configured to regulate the power sent to the implantable medical device based on the power-regulation data received from the implantable medical device.

In another aspect, an implantable medical device system is provided. The implantable medical device system comprises: an implantable medical device, including: an internal radio-frequency receiver, an internal power monitor configured to determine a real-time power requirement of the implantable medical device, and an internal wireless transmitter; and an external device, including: an external radio-frequency transmitter configured to send power signals to the internal radio-frequency receiver via a closely-coupled power link, an external wireless receiver configured to receive power-regulation data from the internal wireless transmitter via a separate data link, where the power-regulation data represents a real-time power requirement of the implantable medical device, and an external power regulator configured to adjust one or more attributes of the power signals based on the power-regulation data received from the implantable medical device.

### BRIEF DESCRIPTION OF THE DRAWINGS

Embodiments of the present invention are described herein in conjunction with the accompanying drawings, in which:

FIG. 1A is a schematic diagram illustrating a cochlear implant system configured to implement aspects of the techniques presented herein;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic view of an exemplary embodiment of an implantable medical device system, in accordance with certain embodiments presented herein;

FIGS. 3A, 3B, and 3C are a series of graphs illustrating aspects of the techniques presented;

FIG. 4 is a flowchart of an exemplary method of power regulation of an implantable medical device, in accordance with certain embodiments presented herein;

FIG. 5 is a block diagram of an implantable stimulation system with which aspects of the techniques presented herein can be implemented.

FIG. 6 is a schematic diagram illustrating a vestibular stimulator system with which aspects of the techniques presented herein can be implemented; and

FIG. 7 is a flowchart of an example method, in accordance with certain embodiments presented herein.

### DETAILED DESCRIPTION

Presented herein are techniques for regulating transmission of power to an implantable medical device via an independent data link. More specifically, in accordance with embodiments presented herein, an external device transmits/sends power to an implantable medical device via a transcutaneous power link (e.g., closely-coupled link). An independent (separate) transcutaneous data link is used by the implantable medical device to provide the external device with information/data representing the real-time (e.g., instantaneous) power requirement of the implantable medical device. The external device controls/regulates the power sent to the implantable medical device based on the data received via the independent data link.

Merely for ease of description, the techniques presented herein are primarily described with reference to a specific implantable medical device system, namely a cochlear implant system. However, it is to be appreciated that the techniques presented herein may also be partially or fully implemented by other types of implantable medical device systems. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, cochlear implants, combinations or variations thereof, etc. The techniques presented herein may also be implemented by other types of implantable medical device systems, including systems comprising dedicated tinnitus therapy devices (tinnitus therapy device systems), vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

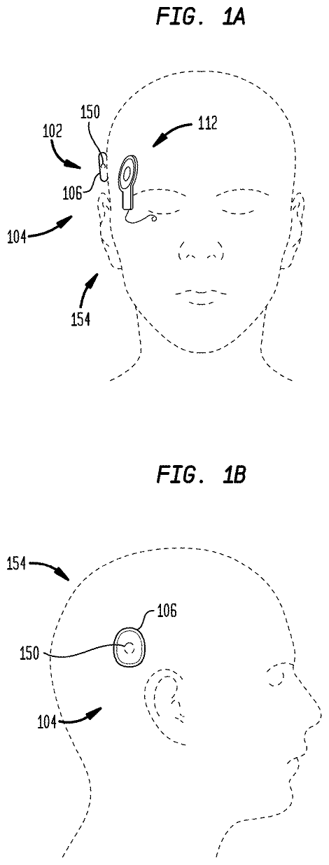

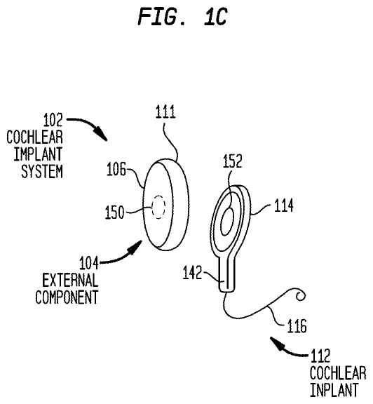

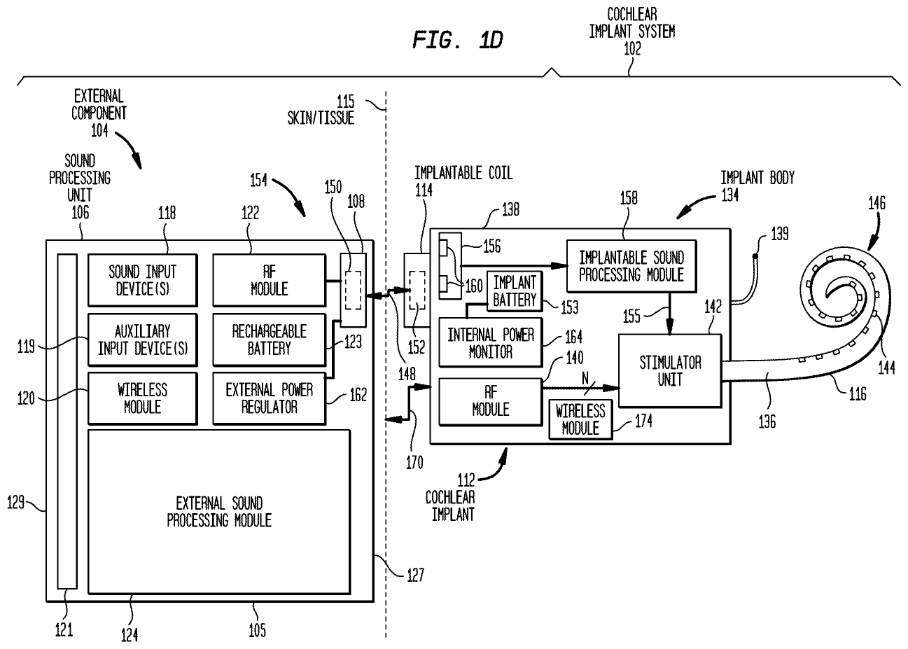

FIGS. 1A-1D illustrate an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component (external device) 104 that is configured to be directly or indirectly attached to the body of the recipient and a cochlear implant 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-ID, the sound processing unit 106 is an off-the-ear (OTE) sound processing unit, sometimes referred to herein as an OTE component, that is configured to send data and power to the cochlear implant 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the cochlear implant 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with cochlear implant 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 1 (e.g., audio ports, such as a Direct Audio Input (DAI), data ports, such as a Universal Serial Bus (USB) port, cable port, etc.), and a wireless transmitter, receiver, and/or transceiver, referred to as a wireless module 120. However, it is to be appreciated that one or more input devices may include additional types of input devices and/or less input devices.

The OTE sound processing unit 106 also comprises the external coil 108, a charging coil 121, a closely-coupled transmitter and/or receiver 122, sometimes referred to as a radio-frequency (RF) transmitter, receiver, and/or transceiver, referred to as RF module 122, at least one rechargeable battery 123, and an external sound processing module 124. The external sound processing module 124 may comprise, for example, one or more processors and a memory device (memory) that includes sound processing logic. The memory device of the external sound processing module 124 may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors of the external sound processing module 124 are, for example, microprocessors or microcontrollers that execute instructions for the sound processing logic stored in the memory device.

The cochlear implant 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF module 140, an implantable sound processing module 158, a stimulator unit 142, an internal power monitor 164, a wireless module 174, and an implantable battery 153 are disposed. The implant body 134 also includes the internal/implantable coil 114 that is generally external to the housing 138, but which is connected to the RF module 140 and/or the internal power monitor 164 via a hermetic feedthrough (not shown in FIG. 1D). In one exemplary embodiment, the internal power monitor 164 may be in direct communication with the internal/implantable coil 114. In an alternative embodiment, the internal power monitor 164 may be in communication with the RF module 140, which is directly connected to the internal/implantable coil 114.

As noted, the stimulating assembly 116 is configured to be at least partially implanted in the recipient's cochlea. The stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

The stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The cochlear implant 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit power and, in certain examples, data, to the cochlear implant 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into processed output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into processed output signals that represent electrical stimulation for delivery to the recipient.

As noted, FIG. 1D illustrates an embodiment in which the external sound processing module 124 in the sound processing unit 106 generates the processed output signals. In an alternative embodiment, the sound processing unit 106 can send less processed information (e.g., audio data) to the cochlear implant 112 and the sound processing operations (e.g., conversion of sounds to processed output signals) can be performed by a processor within the cochlear implant 112.

Returning to the specific example of FIG. 1D, the processed output signals are provided to the RF module 122, which transcutaneously transfers the processed output signals (e.g., in an encoded manner) to the cochlear implant 112 via external coil 108 and implantable coil 114. That is, the processed output signals are received at the RF module 140 via implantable coil 114 and provided to the stimulator unit 142. The stimulator unit 142 is configured to utilize the processed output signals to generate electrical stimulation signals (e.g., current signals) for delivery to the recipient's cochlea. In this way, the cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As noted above, FIGS. 1A-1D illustrate an embodiment in which processed output signals and/or audio data are sent to the cochlear implant 112 via the closely-coupled wireless link 148. It is to be appreciated that this implementation is merely illustrative and that the processed output signals and/or audio data could be sent to the cochlear implant 112 via a separate transcutaneous link, such as a Bluetooth link, Bluetooth Low Energy (BLE) link, a magnetic induction link, a proprietary link, etc.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device of the implantable sound processing module 158 may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors of the implantable sound processing module 158 are, for example, microprocessors or microcontrollers that execute instructions for the sound processing logic stored in the memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in the implantable sound processing module 158 are configured to execute sound processing logic in memory to convert the received input signals into output signals that are provided to the stimulator unit 142. The stimulator unit 142 is configured to utilize the output signals to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, the external component 104 is configured to transmit/send at least power to the cochlear implant 112 via closely-coupled wireless link 148 formed between the external coil 108 with the implantable coil 114. As shown in FIG. 1D, the external component 104 can comprise an external power regulator 162 that is configured to control or regulate the amount of power that the external coil 108 transmits to the implantable coil 114. In one illustrative embodiment, the external power regulator 162 can communicate with the RF module 122 to control attributes (e.g., level) of the power signals transmitted via the closely-coupled wireless link 148.

In addition, the cochlear implant 112 comprises the internal power monitor 164. As described further below, the internal power monitor 164 is configured to determine the real-time (e.g., instantaneous) power requirement of the cochlear implant 112, and communicate the power requirement to the external power regulator 162 via an independent transcutaneous data link 170. Also as described further below, the external power regulator 162 is configured to use the power requirement of the cochlear implant 112, as received via the independent data link 170, to regulate the amount of power that the external coil 108 transmits to the implantable coil 114.

The independent data link 170 is a wireless communication link formed between wireless module 174 of cochlear implant 112 and wireless module 120 of external component 104. The independent data link 170 can be, for example, a short-range communication link, such as a Bluetooth link, a Bluetooth Low Energy (BLE) link, a proprietary link, etc.

As noted, implantable medical devices, such as cochlear implants, generally operate based on power received from an external device. The received power can be used to operate the implantable medical device and/or to recharge an implantable battery within the medical device. The power is generally transferred from the external device to the implantable medical device via a closely-coupled transcutaneous link, sometimes referred to herein as a “power link.” As used herein, a power link can include only power signals or can include both power and data signals. The data signals can be, for example, modulated onto the power signals, separated in time from the power signals, etc.

The amount of power required by an implantable medical device at any given time can change. That is, the real-time (e.g., instantaneous) power requirement of the implantable medical device varies, over time, potentially based on a number of different factors, such as the ambient environment (e.g., ambient sound environment), implant load, stimulation parameters, implantable battery charge status, etc. Due to the variable amount of power that an implantable device requires, it is preferable to regulate or control, in real-time, the amount of power that the external device transfers to the implantable device. For example, the transfer of insufficient power to the implantable medical device can render the implantable medical device partially or fully inoperable, limit battery charging, etc. Conversely, the transfer of extraneous power is not only inefficient, but could also damage the implantable medical device. In particular, surplus power that is transferred to the implantable device may be dissipated as unnecessary heat, meaning that power is wasted. This heat can also lead to a shorter battery life of the external and/or implantable batteries, cause discomfort, pain, or injury to the recipient of the implantable device, etc.

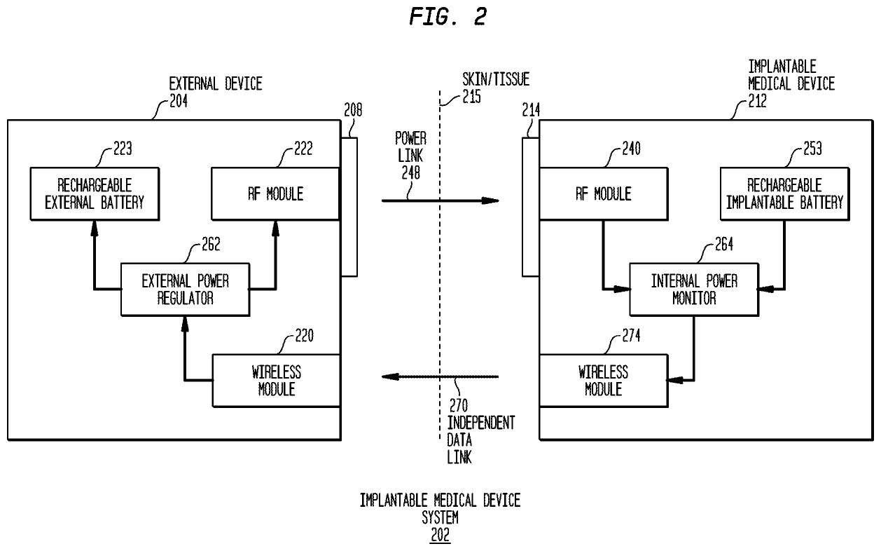

In accordance with embodiments presented herein, an external device is configured to regulate the power transferred to an implantable medical device based on information/data received from the implantable medical device. The data received from the implantable medical device indicates/represents the real-time power requirement of the implantable medical device and is received via a wireless link that is independent (separate) from the power link. This wireless link that is independent (separate) from the power link is sometimes referred to herein as an “independent data link” or “independent back-link” from the implantable medical device to the external device. FIG. 2 is a block diagram illustrating further details of the techniques presented herein.

More specifically, shown in FIG. 2 illustrates an example implantable medical device system 202 comprising an implantable medical device (implantable component) 212 and an external device (external component) 204. The external device 204 comprises an external rechargeable battery 223, an RF module 222, an external coil 208, an external power regulator 262, and a wireless module 220. The implantable medical device 212 comprises an implantable rechargeable battery 253, an RF module 240, an implantable coil 214, an internal power monitor 262, and a wireless module 274.

It is to be appreciated that, for ease of illustration, FIG. 2 only illustrates components/elements of the external device 204 and of the implantable medical device 212 that are relevant to the techniques described herein. As such, it is to be appreciated that the external device 204 and the implantable medical device 212 can include a number of other components, or less components (e.g., rechargeable external battery 223 and or rechargeable implantable battery 252 could be omitted) and each can have a number of different arrangements. For example, the external device 204 could be an external processing device, stand-alone charging device, etc., while the implantable medical device 212 could be a cochlear implant or other type of auditory prosthesis systems that include one or more other types of auditory prostheses, a tinnitus therapy device, a vestibular device, a visual device, a sensor, a pacemaker, a drug delivery system, a defibrillator, a functional electrical stimulation device, a seizure device, (e.g., a device for monitoring and/or treating epileptic events), a sleep apnea devices, an electroporation device, etc.

In operation, the RF module 222 in the external device 204 is configured to transfer power from the rechargeable battery 223 to the RF module 240 via a closely-coupled power link 248 formed via external coil 208 and implantable coil 214. That is, the external device 204 sends power signals to the implantable medical device 212 via the power link 248. The power received at the RF module 240 can be used to, for example, power operation of the implantable medical device 212 and/or to recharge the rechargeable implantable battery 253.

The internal power monitor 264 is configured to monitor the real-time power requirement of the implantable medical device 212. The real-time power requirement of the implantable medical device 212 can vary based on a number of different factors, such as charge level, voltage, or status of the rechargeable implantable battery 253 (e.g., a minimum battery voltage level to ensure the implantable medical device remains powered, a maximum voltage level above which results in a shunt that releases extraneous power via heat), an amount of power consumed by the implantable medical device at a given time (e.g., an electrical load or electrical stimulation voltage requirements of the implant), compliance requirements of the implant device (e.g., a compliance voltage), etc.

Stated differently, the internal power monitor 264 is configured to determine the real-time (e.g., instantaneous) power requirement of the implantable medical device 212. The internal power monitor 264 is configured to send data/information representing the real-time power requirement of the implantable medical device 212 to the external power regulator 262 via an independent transcutaneous data link 270 (i.e., a wireless link that is separate from the power link 248). The independent data link 270 is a wireless communication link formed between wireless module 274 of the implantable medical device 212 and the wireless module 220 of the external component 204. The independent data link 270 can be, for example, a short-range communication link, such as a Bluetooth link, a Bluetooth Low Energy (BLE) link, a proprietary link, etc.

The external power regulator 262 is configured to use the power requirement of the implantable medical device 212, as represented in the data received via the independent data link 270, to regulate the amount of power sent from the external device 204 to the implantable medical device 212. For example, in one illustrative embodiment, the external power regulator 262 can communicate with the RF module 222 to control attributes (e.g., level) of the power signals transmitted via the power link (closely-coupled RF link) 248.

The internal power monitor 264 and the external power regulator 262 can have a number of different arrangements and can be implemented in hardware, software, and or/combinations thereof. For example, the internal power monitor 264 and/or the external power regulator 262 can comprise, for example, one or more processors and a memory device (memory) that includes power monitoring or power regulation logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors, microcontrollers, digital signal processors (DSPs), etc. that execute instructions for the power regulation logic stored in the memory device.

The independent data link 270 is independent and separate from the power link 248 at least because the two links use two different and spaced carrier frequencies. The independent data link 270 is used to send data from the implantable medical device 212 to the external device 204, and this data at least includes, indicates, or otherwise represents the real-time power requirement of the implantable medical device. That is, the data sent on the independent data link 270 includes data useable to regulate, control, or otherwise adjust the amount of power sent by the external device 204 to the implantable medical device 212 via the power link 248. As the power requirement of the implantable medical device 212 changes, the data sent on the independent data link 270 will also change to communicate the changing power requirement of the implantable medical device. The external device 204 (e.g., external power regulator 262) will accordingly use the data to determine an appropriate amount of power for transfer to the implantable medical device 212 via the power link 248.

In certain embodiments, the power link 248 utilizes a continuous carrier signal/wave. In certain embodiments, data is not encoded, sent, or received on the power link 248. While independent data link 270 is used to transmit/receive data, the carrier signal of the power link 248 is used to transfer power at appropriate levels using an appropriate amplitude of the carrier signal corresponding to the real-time power requirement of the implantable medical device.

In one embodiment, the power link 248 is only used as a forward-link to transfer power from the external device 204 to the implantable medical device 212, and independent data link 270 is only used as a back-link to transmit “power-regulation data” (e.g., data/information representing the real-time power requirement of the implantable medical device 212) from the implantable medical device 212 to the external device 204. In an alternative embodiment, however, independent data link 270 is used as both a back-link and a forward-link, i.e., independent data link 270 is configured to facilitate bidirectional communication between the implantable medical device 212 and the external device 204. For example, in the case of a hearing aid, cochlear implant, or other hearing device, external device 204 may have a microphone and the independent data link 270 may function as a forward-link to transmit audio data from the external device 204 to the implantable medical device 212 in addition to functioning as a back-link to send power-regulation data from the implantable medical device 212 to the external device 204. The information transmitted using the forward-link and/or the back-link of independent data link 270 is not limited to audio data and power-regulation data. Independent data link 270 may be used to transfer any data including but not limited to data relating to environmental/sensory input and/or device characteristics.

In operation, the power link 248 and independent data link 270 use carrier frequencies that are substantially far apart from one another on the electromagnetic spectrum such that the links have no or minimal electromagnetic interference there between. In one exemplary embodiment, the carrier frequencies of the power link 248 and independent data link 270 are at least an order of magnitude apart. In one specific such example, the power link 248 may use a 6.78 MHz carrier frequency and independent data link 270 may use a 2.4 GHz carrier frequency, however, any two different carrier frequencies, which preferably do not substantially interfere, may be used for the power link 248 and independent data link 270.

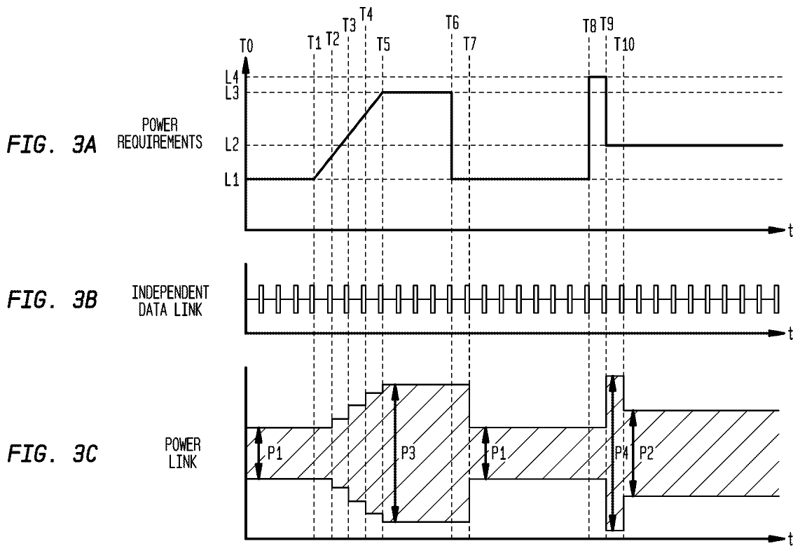

FIGS. 3A, 3B, and 3C are a series of graphs illustrating aspects of the techniques presented. FIG. 3A is a graph illustrating variable power requirement of implantable medical device 212 over time. FIG. 3B is a graph illustrating communication signals sent from the implantable medical device 212 to the external device 204 via independent data link 270, while FIG. 3C is a graph illustrating power signals sent from external device 204 to the implantable medical device 212 via power link 248. For ease of description, the examples of FIGS. 3A, 3B, and 3C will be described with reference to implantable medical device system 202 of FIG. 2.

As noted, the power requirement of an implantable medical device, such as implantable medical device 212, can vary over time. This is generally shown in FIG. 3A. FIG. 3B illustrates a timing of transmission/sending of data from the implantable medical device 212 to the external device 204. The data transmitted from the implantable medical device 212 to the external device 204 includes at least power-regulation data (e.g., data/information representing the real-time power requirement of the implantable medical device 212). In certain examples, the implantable medical device 212 can send data packets at regular time intervals such as, e.g., at a predetermined frequency. Referring to FIG. 3B, each pulse (vertical rectangle) represents a communication packet or a window during which communication packets are sent/exchanged. Further, packets are sent/exchanged at regular intervals, and the period is equal to the difference between time T2 and time T1. In one embodiment, the internal power monitor 264 determines the instantaneous power requirement of the implantable medical device 212 at the time of or shortly before the transmission of the packet or shortly before the window during which communication packets are sent/exchanged, and the determined power requirement are then transmitted to the external device 204.

In an exemplary embodiment, as shown in FIG. 3C, the power transferred to the implantable medical device 212 by the external device 204 corresponds to a magnitude of the power link signal and is a function of the power-regulation data received by the external device 204 from the implantable medical device 212 via the independent data link 270. More specifically, as shown in FIGS. 3A and 3C, from time T0 to time T1, the power requirement of the implantable medical device 212 initially remains at power requirement level L1 and a magnitude of the power link signal remains at power transmission level P1. Data packets transmitted between time T0 and time T1 on the independent data link 270 provide an indication to the external device 204 that the power requirement of the implantable medical device 212 remain constant at power requirement level L1. From time TI to time T5, the power requirement of the implantable medical device 212 gradually increases from power requirement level L1 to power requirement level L3. During this time, data packets transmitted via the independent data link 270 communicate the increased power requirement to the external device 204. Upon receipt of the progressively increasing power requirement, which specifically occurs at times T2, T3, T4, and T5 when a respective packet is received or each transmission/exchange window terminates, the external device 204 (external power regulator 262) increases the magnitude of the power link signal stepwise at times T2, T3, T4, and T5. At time T5, the power requirement of the implantable medical device 212 is at power requirement level L3 and the magnitude of the power link signal is at power transmission level P3.

From time T5 to time T6, the power requirement of the implantable medical device 212 remains at power requirement level L3 and data packets transmitted via independent data link 270 between time T5 and time T6 provide an indication to the external device 204 that the power requirement of the implantable medical device 212 remains constant at power requirement level L3. As such, the magnitude of the power link signal transmitted to the implantable medical device 212 remains at power transmission level P3.

At time T6, the power requirement of the implantable medical device 212 instantaneously or nearly instantaneously decreases from power requirement level L3 to power requirement level L1. As noted above, this change in power requirement is communicated to the external device in one or more packets sent on independent data link 270. At time T7, at which time the packet(s) indicating the power requirement change have already been received at the external device, the magnitude of the power link signal transmitted to the implantable medical device 212 changes from power transmission level P3 to power transmission level P1.

From time T7 to time T8, the power requirement of the implantable medical device 212 remains at power requirement level LI and packets transmitted via independent data link 270 between time T7 and time T8 provide an indication to the external device that the power requirement of the implantable medical device 212 remains constant at power requirement level L1. As a result, the magnitude of the power link signal transmitted to the implantable medical device 212 remains at power transmission level P1.

At time T8, the power requirement of the implantable medical device 212 instantaneously or nearly instantaneously increases from power requirement level LI to power requirement level L4. At time T9, at which time the packet(s) indicating the power requirement change have already been received at the external device, the magnitude of the power link signal transmitted to the implantable medical device 212 changes from power transmission level P1 to power transmission level P4.

At time T9, the power requirement of the implantable medical device 212 instantaneously or nearly instantaneously decreases from power requirement level L4 to power requirement level L2. At time T10, at which time the packet(s) indicating the power requirement change have already been received at the external device, the magnitude of the power link signal transmitted to the implantable medical device 212 changes from power transmission level P4 to power transmission level P2.

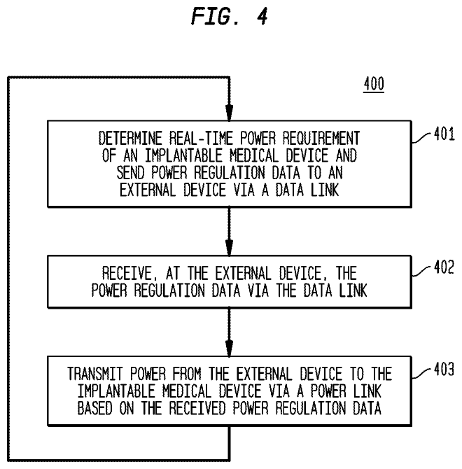

FIG. 4 is a flowchart of an exemplary method 400 for power regulation of an implantable medical device, in accordance with certain embodiments presented herein. For ease of illustration, the example of FIG. 4 will again be described with reference to the arrangement of FIG. 2.

At step 401, the implantable medical device 212 determines the real-time power requirement of the implantable medical device and sends power-regulation data (e.g., data/information representing the real-time power requirement of the implantable medical device 212) to the external device 204 via the independent data link 270. At step 402, the external device 204 receives the power regulation data from the implantable medical device 212 via the independent data link 270. At step 403, the external device 204 transmits power to the implantable medical device via the power link 248, where attributes (e.g., level or magnitude) of the power signals are based on the received power-regulation data. As noted above, the power link 248 may be used for the sole purpose of transmitting power and the independent data link 270 may be used for the sole purpose of transmitting and/or receiving data. Furthermore, also as noted above, the independent data link 270 and the power link 248 are independent and separate in operation and functionality. In this regard, the independent data link 270 and the power link 248 may, e.g., operate simultaneously and using different radio frequencies that have little to no radio frequency interference there between.

In certain embodiments, the implantable medical device 212 can continuously or periodically transmit the real-time (e.g., instantaneous) power requirement to the external device 204. In an alternative embodiment, the implantable medical device 212 only communicates with the external device 204 when the power requirement changes. When the power requirement of the implantable medical device 212 changes, the implantable medical device 212 and the external device 204 communicate with updated power requirement information. As such, the frequency of communications between the implantable medical device 212 and the external device 204, as well as the content of the power-regulation data, may vary depending on the configuration of the system.

After step 404, the process may return to step 402 where the implantable medical device 212 can again transmit to the power-regulation data of the implantable medical device via the data link 270. The operations of 402 and 404 can continue during operation of the implantable medical device 212 with the external device 204.

It is to be appreciated that method 400 illustrated in FIG. 4 is not necessarily limited to the external device 204 receiving power-regulation data from the implantable medical device 212 before the external device transmits power to the implantable medical device. That is, the external device 204 can transmit power to the implantable medical device 212 before the implantable medical device 212 transmits power-regulation data to the external device.

It is also to be appreciated that the device that determines the amount of power transmitted from the external device 204 to the implantable medical device is not necessarily limited. For example, the implantable medical device 212, the external device 204, or another device that communicates with the implantable medical device and/or the external device, or any of these devices alone or in combination, may make the determination of the appropriate amount of power that is transmitted from the external device to the implantable medical device for optimal power transfer. In this regard, the power-regulation data transmitted by the implantable medical device 212 is not limited and can have a number of different forms. For example, the implantable medical device 212 may be the device that determines the appropriate amount of power to be transmitted to itself from the external device 204, and the power-regulation data may indicate the appropriate amount of power. In the alternative, the external device 204 may be the device that determines the appropriate amount of power to be transmitted to the implantable medical device, and the power-regulation data transmitted from the implantable medical device may be data used by the external device 204 to determine the appropriate amount of power to transmit to the implantable medical device 212 via the power link 248. In yet another embodiment, a device separate from the external device 212, such as a smartphone, smart watch, tablet computer, computer, etc., which may or may not be on or near the recipient, may communicate with the implantable medical device 212 and/or the external device 204 to make the determination or aid in making the determination of the appropriate amount of power to transmit to the implantable medical device from the external device. As such, the data link 270 and the power-regulation data transmitted on the data link may include communications to the external device 204 and/or communications to another device separate from the external device. As such, the power power-regulation data transmitted via the data link 270 may be any information that is used to regulate the power transmitted via the power link.

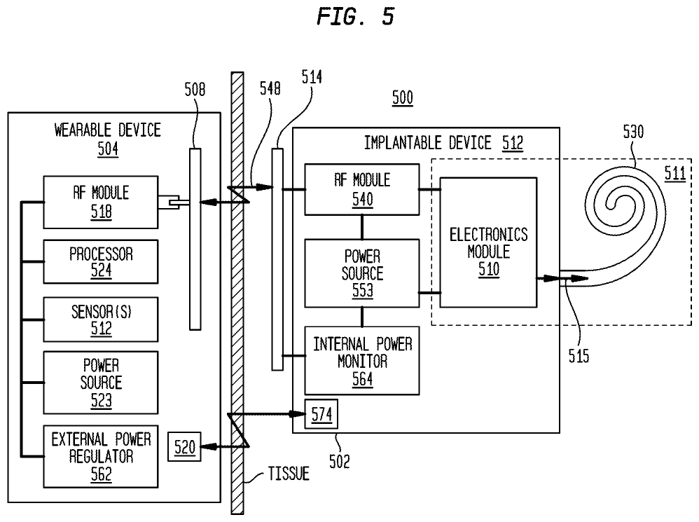

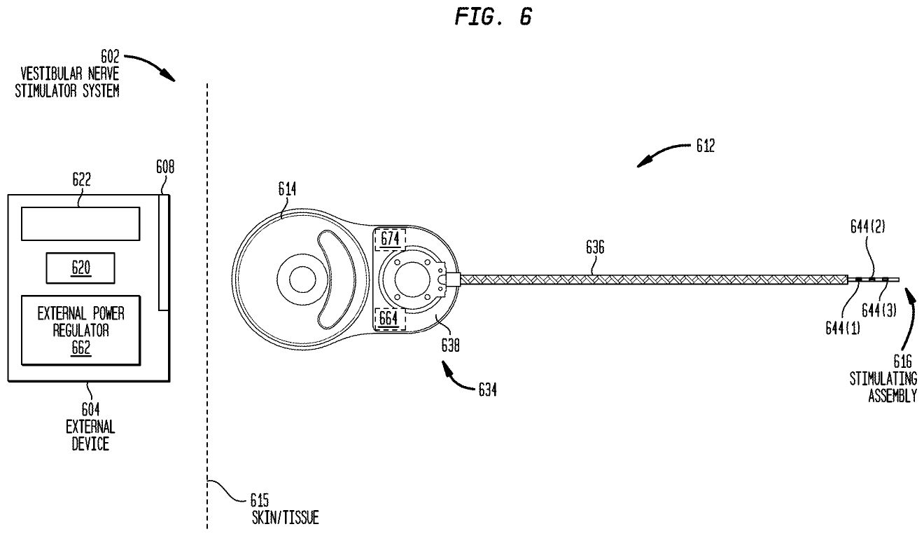

As previously described, the technology disclosed herein can be applied in any of a variety of circumstances and with a variety of different devices. Example devices that can benefit from technology disclosed herein are described in more detail in FIGS. 5 and 6, below. As described below, the operating parameters for the devices described with reference to FIGS. 5 and 6 may be configured using power regulation system(s) analogous to the power regulation system(s) described with reference to FIGS. 1-4. For example, the techniques described herein can be used to, e.g., optimize the efficiency of power transfer to wearable medical devices, such as an implantable stimulation system as described in FIG. 5, a vestibular stimulator as described in FIG. 6, a retinal prosthesis, etc. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 5 is a functional block diagram of an implantable stimulator system 500 that can benefit from the technologies described herein. The implantable stimulator system 500 includes the wearable device 504 acting as an external processor device and an implantable device 512 acting as an implanted stimulator device. In examples, the implantable device 512 is an implantable stimulator device configured to be implanted beneath a recipient's tissue (e.g., skin). In examples, the implantable device 512 includes a biocompatible implantable housing 502. Here, the wearable device 504 is configured to transcutaneously couple with the implantable device 512 via a wireless connection to provide additional functionality to the implantable device 512.

In the illustrated example, the wearable device 504 includes one or more sensors 512, a processor 524, an RF module 518, a power source 523, a coil 508, a wireless module 520, and an external power regulator 562. The one or more sensors 512 can be one or more units configured to produce data based on sensed activities. In an example where the stimulation system 500 is an auditory prosthesis system, the one or more sensors 512 include sound input sensors, such as a microphone, an electrical input for an FM hearing system, other components for receiving sound input, or combinations thereof. Where the stimulation system 500 is a visual prosthesis system, the one or more sensors 512 can include one or more cameras or other visual sensors. Where the stimulation system 500 is a cardiac stimulator, the one or more sensors 512 can include cardiac monitors. The processor 524 can be a component (e.g., a central processing unit) configured to control stimulation provided by the implantable device 512. The stimulation can be controlled based on data from the sensor 512, a stimulation schedule, or other data. Where the stimulation system 500 is an auditory prosthesis, the processor 524 can be configured to convert sound signals received from the sensor(s) 512 (e.g., acting as a sound input unit) into signals transmitted/sent to the implantable device via a closely-coupled link 548. Stimulation signals can be generated by the processor 524 and transmitted, using the RF module 518, to the implantable device 512 for use in providing stimulation.

In the illustrated example, the implantable device 512 includes an RF module 540, a power source 553, and a medical instrument 511 that includes an electronics module 510 and a stimulator assembly 530. The implantable device 512 further includes a hermetically sealed, biocompatible implantable housing 502 enclosing one or more of the components.

The electronics module 510 can include one or more other components to provide medical device functionality. In many examples, the electronics module 510 includes one or more components for receiving a signal and converting the signal into the stimulation signal 515. The electronics module 510 can further include a stimulator unit. The electronics module 510 can generate or control delivery of the stimulation signals 515 to the stimulator assembly 530. In examples, the electronics module 510 includes one or more processors (e.g., central processing units or microcontrollers) coupled to memory components (e.g., flash memory) storing instructions that when executed cause performance of an operation. In examples, the electronics module 510 generates and monitors parameters associated with generating and delivering the stimulus (e.g., output voltage, output current, or line impedance).

The stimulator assembly 530 can be a component configured to provide stimulation to target tissue. In the illustrated example, the stimulator assembly 530 is an electrode assembly that includes an array of electrode contacts disposed on a lead. The lead can be disposed proximate tissue to be stimulated. Where the system 500 is a cochlear implant system, the stimulator assembly 530 can be inserted into the recipient's cochlea. The stimulator assembly 530 can be configured to deliver stimulation signals 515 (e.g., electrical stimulation signals) generated by the electronics module 510 to the cochlea to cause the recipient to experience a hearing percept. In other examples, the stimulator assembly 530 is a vibratory actuator disposed inside or outside of a housing of the implantable device 512 and configured to generate vibrations. The vibratory actuator receives the stimulation signals 515 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion. The RF module 540 can be components configured to transcutaneously receive and/or transmit a signal (e.g., a power signal and/or a data signal) via closely-coupled link 548. The RF module 540 can be a collection of one or more components that form part of a transcutaneous energy or data transfer system to transfer the signal between the wearable device 504 and the implantable device 512 via closely-coupled link 548. Various types of signal transfer, such as electromagnetic, capacitive, and inductive transfer, can be used to usably receive or transmit the signals via closely-coupled link 548. The RF module 540 can include or be electrically connected to a coil 514.

As illustrated, the wearable device 504 includes a coil 508 for transcutaneous transfer of signals with the implantable coil 514 via closely-coupled link 548. As noted above, the transcutaneous transfer of signals between coil 508 and the coil 514 can include the transfer of power and/or data from the coil 508 to the coil 514. The power source 523 and/or power source 553 can be one or more components configured to provide operational power to other components. The power source 523 and/or power source 553 can be or include one or more rechargeable batteries. Power for the batteries can be received from a source and stored in the battery. The power can then be distributed to the other components as needed for operation.

In the illustrated example, the implantable device 512 further includes an internal power monitor 564, which may be similar to internal power monitor 564 described above, and a wireless module 574. The internal power monitor 564 is configured to determine the real-time (e.g., instantaneous) power requirement of the implantable device 512, and communicate the power requirement to the external power regulator 562 via an independent transcutaneous data link 570 formed between wireless modules 574 and 520. The external power regulator 562 is configured to use the power requirement of the implantable device 512, as received via the independent data link 570, to regulate the amount of power that the wearable device 504 transmits to the implantable device 512.

As should be appreciated, while particular components are described in conjunction with FIG. 5, technology disclosed herein can be applied in any of a variety of circumstances. The above discussion is not meant to suggest that the disclosed techniques are only suitable for implementation within systems akin to that illustrated in and described with respect to FIG. 5. In general, additional configurations can be used to practice the methods and systems herein and/or some aspects described can be excluded without departing from the methods and systems disclosed herein.

FIG. 6 illustrates an example vestibular stimulator system 602, with which embodiments presented herein can be implemented. As shown, the vestibular stimulator system 602 comprises an implantable component (vestibular stimulator) 612 and an external device/component 604 (e.g., external processing device, battery charger, remote control, etc.). The external device 604 comprises an RF module 660, an external coil 608, a wireless module 620, and an external power regulator 662.

The vestibular stimulator 612 comprises an implant body (main module) 634, a lead region 636, and a stimulating assembly 616, all configured to be implanted under the skin/tissue (tissue) 615 of the recipient. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the RF interface circuitry via a hermetic feedthrough (not shown). The implant body 634 further comprises an internal power monitor 664 and a wireless module 674.

The stimulating assembly 616 comprises a plurality of electrodes 644(1)-(3) disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the recipient's vestibular system.

The stimulating assembly 616 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 612, the external device 604, and/or another external device, can be configured to implement the techniques presented herein. That is, the vestibular stimulator 612, possibly in combination with the external device 604 and/or another external device, can include a system that optimizes power transfer using an independent data link, as described elsewhere herein. In particular, the external device 604 is configured to transfer power to the vestibular stimulator 612 via a power link 648 formed between coils 608 and 614. The internal power monitor 664 is configured to determine the real-time (e.g., instantaneous) power requirement of the vestibular stimulator 612, and communicate the power requirement to external power regulator 662 via an independent transcutaneous data link 670 formed between wireless modules 674 and 620. The external power regulator 662 is configured to use the power requirement of the vestibular stimulator 612, as received via the independent data link 670, to regulate the amount of power that the external device 604 transmits to the vestibular stimulator 612.

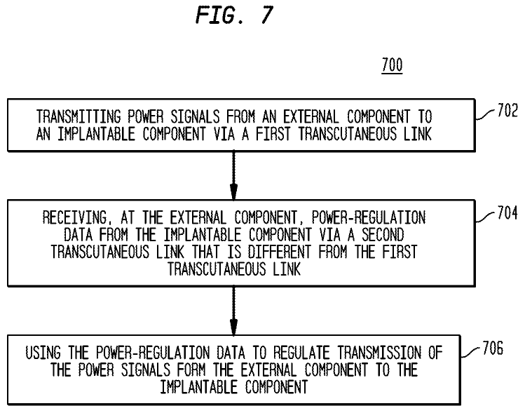

FIG. 7 is a flowchart of a method 700, in accordance with certain embodiments presented herein. Method 700 begins at 702 where an external component transmits/sends power signals to an implantable component via a first transcutaneous link. At 704, the external component receives power-regulation data from the implantable component via a second transcutaneous link that is different from the first transcutaneous link. At 706, the external component uses the power-regulation data to regulate transmission of the power signals from the external component to the implantable component.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
transmitting power from an external device to an implantable medical device via a power link; and
receiving, at the external device, data from the implantable medical device via a data link that is separate from the power link,
wherein the data indicates a real-time power requirement of the implantable medical device.

2. The method of claim 1, further comprising:
regulating the power transmitted via the power link using the real-time power requirement of the implantable medical device indicated in the data received from the implantable medical device.

3. The method of claim 2, wherein regulating the power transmitted via the power link comprises:
changing an amplitude of a carrier signal of the power link based on the real-time power requirement of the implantable medical device.

4. The method of claim 2, wherein the real-time power requirement is associated with a compliance voltage of the implantable medical device.

5. The method of claim 2, wherein the real-time power requirement is associated with a load of the implantable medical device.

6. The method of claim 5, further comprising:
sensing the load at the implantable medical device; and
generating the data based on the sensing of the load.

7. The method of claim 1, wherein transmitting the power to the implantable medical device via the power link comprises:
transmitting only power via a first radio frequency channel.

8. The method of claim 1, wherein transmitting the power to the implantable medical device via the power link comprises:
transmitting power and data via a first radio frequency channel.

9. The method of claim 1, wherein transmitting the power to the implantable medical device via the power link comprises:
using a continuous carrier signal to transmit the power.

10. The method of claim 9, wherein the continuous carrier signal is unmodulated.

11. The method of claim 1, wherein receiving the data from the implantable medical device via a data link comprises:
receiving the data at a frequency that is different from a frequency of the power link, wherein the difference in frequency is such that spectrums of the power link and the data link do not interfere with each other.

12. The method of claim 1, wherein transmitting the power to the implantable medical device via the power link comprises:
transmitting the power via a closely-coupled link operating at a frequency of approximately 6.78 megahertz (MHz), and wherein receiving the data from the implantable medical device via a communications link comprises:
receiving the data at a frequency that is approximately 2.4 Gigahertz (GHz).

13. (canceled)

14. The method of claim 1, wherein the power link is a closely-coupled inductive link.

15. The method of claim 1, wherein the external device is a sound processor configured to transfer power and data to the implantable medical device.

16. The method of claim 15, wherein the external device is an external charger configured to at least transfer power to the implantable medical device.

17. The method of claim 1, wherein the implantable medical device is at least one of a cochlear implant or a bone conduction hearing implant.

18-34. (canceled)

35. An apparatus, comprising:
a radio-frequency module configured to send power to an implantable medical device via a closely-coupled power link;
a wireless module configured to receive power-regulation data from the implantable medical device, where the power-regulation data is received via a data link that is separate from the closely-coupled power link; and
an external power regulator configured to regulate the power sent to the implantable medical device based on the power-regulation data received from the implantable medical device.

36. The apparatus of claim 35, wherein the power-regulation data indicates a real-time power requirement of the implantable medical device.

37. The apparatus of claim 35, wherein the external power regulator is configured to change an amplitude of a carrier signal of the closely-coupled power link based on the power-regulation data.

38. The apparatus of claim 35, wherein the power-regulation data is associated with a compliance voltage of the implantable medical device.

39. The apparatus of claim 35, wherein the power-regulation data is associated with a load of the implantable medical device.

40. The apparatus of claim 35, wherein the radio-frequency module is configured to transmit only power to the implantable medical device via a first radio frequency channel.

41. The apparatus of claim 35, wherein the radio-frequency module is configured to transmit power and data to the implantable medical device via a first radio frequency channel.

42. The apparatus of claim 35, wherein the radio-frequency module is configured to transmit power to the implantable medical device using a continuous carrier signal.

43. The apparatus of claim 35, wherein the closely-coupled power link and the data link are frequency spaced from one another such that that data link does not interfere with operation of the closely-coupled power link.

44. The apparatus of claim 35, wherein the closely-coupled power link has a frequency that is approximately 6.78 megahertz (MHz), and wherein the data link has a frequency that is approximately 2.4 Gigahertz (GHz).

45. (canceled)

46. The apparatus of claim 35, wherein the apparatus is a sound processor configured to transfer power and data to the implantable medical device.

47. The apparatus of claim 35, wherein the apparatus is an external charger configured to at least transfer power to the implantable medical device.

48. The apparatus of claim 35, wherein the implantable medical device is at least one of a cochlear implant or a bone conduction hearing implant.

49. (canceled)
